(19) United States
(12) Patent Application Publication
(10) Pub. No.: US 2010/0068141 A1
(43) Pub. Date: Mar. 18, 2010

(54) USE OF HEAT SHOCK TO TREAT OCULAR DISEASE

(75) Inventors: Shalesh Kaushal, Gainesville, FL (US); Maria G. Grant, Archer, FL (US)

Correspondence Address: EDWARDS ANGELL PALMER & DODGE LLP, P.O. BOX 55874, BOSTON, MA, 02205, US

(73) Assignee: UNIVERSITY OF FLORIDA, Gainesville, FL (US)

(21) Appl. No.: 11/989,347

(22) PCT Filed: Jul. 27, 2006
(86) PCT No.: PCT/US2006/029392

§ 371 Date: Dec. 3, 2009

Related U.S. Application Data
(60) Provisional application No. 60/703,068, filed on Jul. 27, 2005.
(60) Provisional application No. 60/729,182, filed on Oct. 21, 2005.

(51) Int. Cl. A61K 38/00; A61K 31/395; A61K 31/365; A61K 48/00; A61P 27/02; A61P 27/06; A61K 38/22; A61K 35/12; A61K 49/00; A61F 9/008

(52) U.S. Cl. 424/9.1; 514/183; 514/450; 514/2; 514/44 R; 424/85.1; 424/93.7; 606/4

(57) ABSTRACT

The invention generally provides methods for recruiting stem cells to an ocular tissue. The methods involve inducing heat shock in the ocular tissue using a subthreshold laser and/or an agent. In some embodiments, the heat shock is induced following the administration of an agent that mobilizes HSCs.

### CROSS-REFERENCE TO RELATED APPLICATIONS

This application claims the benefit of the following U.S. Provisional Application Nos. 60/703,068, which was filed on Jul. 27, 2005, and 60/729,182, which was filed on Oct. 21, 2005; the entire contents of each of these applications is hereby incorporated by reference.

### STATEMENT OF RIGHTS TO INVENTIONS MADE UNDER FEDERALLY SPONSORED RESEARCH

This work was supported by a National Eye Institute Grant, Grant No. EY016070-01. The government may have certain rights in the invention.

### BACKGROUND OF THE INVENTION

Age-related macular degeneration (ARMD) is the most common cause of blindness in the Western world. As a consequence, ARMD is an important public health problem. Approximately 85 to 90% of patients have the non-exudative (dry) form of ARMD. This consists of retinal pigment epithelium atrophy, depigmentation and retinal pigment epithelium loss. Dry ARMD affects the elderly population, seriously compromising the quality of their lives. Treatment options have been limited. Outside of nutritional and vitamin supplements, there is no effective specific treatment for form of the disease. Current therapeutic approaches for treating ARMD are ineffective. Thus, improved therapeutic methods are urgently required.

### SUMMARY OF THE INVENTION

As described below, the present invention features methods of treating an ocular disease by inducing a heat shock response that recruits stem cells to the eye to repair damaged tissue.

In a first aspect, the invention features a method for ameliorating an ocular disease in a subject. The method involves inducing heat shock in at least one cell of an ocular tissue; and recruiting a stem cell to the ocular tissue, thereby ameliorating the ocular disorder. In one embodiment, the heat shock is induced in the ocular tissue using sub-visible threshold laser (SVL) stimulation. In another embodiment, the heat shock is induced using a small compound selected from the group consisting of geldanamycin, celastrol, 17-allylamino-17-demethoxygeldanamycin, EC102, radicicol, geranylgeranylacetone, paeoniflorin, PU-DZ8, and H-71. In yet another embodiment, heat shock is induced using a heat shock polypeptide or an expression vector containing a polynucleotide encoding a heat shock polypeptide (e.g., Hsp100, Hsp90, Hsp70, Hsp60, and Hsp40). In yet another aspect, the heat shock polypeptide is Hsp70 or Hsp90. In yet another embodiment, the stem cell is a bone marrow derived cell, such as a hematopoietic stem cell. In yet another embodiment, the method reduces at least one symptom of the ocular disease or disorder. In further embodiments, the ocular disease or disorder is any one or more of diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy.

In yet another aspect, the invention provides a method of recruiting a stem cell to an ocular tissue of a subject in need thereof. The method involves stimulating the ocular tissue with a sub-threshold laser, wherein the level of stimulation is sufficient to recruit at least one stem cell to the tissue. In various embodiments, the laser treatment features a 10% or 15% duty cycle is used; the sub-threshold laser has a wavelength from at least about 100 nm to 2000 nm (e.g., 100, 200, 250, 300, 500, 750, 1000, 1250, 1500, 1750, 2000). In yet other embodiments, the sub-threshold laser energy is from about 5 mW to 200 mW (e.g., 5, 10, 25, 50, 75, 100, 125, 150, 175, 200 mW) and is administered in a micropulse having a duration from about 0.001 msec to 1.0 msec (e.g., 0.001, 0.005, 0.01, 0.025, 0.5, 0.75, or 1.0 msec). In other embodiments, the laser energy is between about 10 mW to 100 mW and the duration of the micropulse is 0.1 msec. In still other embodiments, the stimulation increases the expression or biological activity of a heat shock protein selected from the group consisting of Hsp100, Hsp90, Hsp70, Hsp60, and Hsp40. In other embodiments, the increase is by at least about 5, 10, 20, 25, 50, 75, or 100% or more. In still other embodiments, the stimulation alters the expression or activity of a protein selected from the group consisting of SDF-1, VEGF, HIF-1α, crystallin, hypoxia-inducible factor 1-alpha (HIF-1a), and CXCR-4. In still other embodiments, the method increases the expression of an Hsp70 or Hsp90 polypeptide by at least 10-fold, 20-fold, 40-fold, 50-fold, or more.

In yet another aspect, the invention provides a method of recruiting a stem cell to an ocular tissue of a subject in need thereof. The method involves administering an agent to a subject in an amount sufficient to induce heat shock in an ocular tissue; and recruiting a stem cell to the ocular tissue.

In yet another aspect, the invention provides a method of ameliorating an ocular disease or disorder in a subject in need thereof. The method involves administering an agent to a subject in an amount sufficient to induce heat shock in an ocular tissue; and recruiting a stem cell to the ocular tissue, thereby ameliorating the ocular disease or disorder.

In a related aspect, the invention provides a method of regenerating the retina in a subject in need thereof. The method involves administering an agent to a subject in an amount sufficient to induce heat shock in an ocular tissue; and recruiting a stem cell to the ocular tissue, thereby regenerating the retina.

In another related aspect, the invention provides a method of repairing retinal pigment epithelium damage in a subject in need thereof. The method involves administering an agent to a subject in an amount sufficient to induce heat shock in an ocular tissue; and recruiting a stem cell to the ocular tissue, thereby repairing the retinal pigment epithelium.

In yet another aspect, the invention features a method of ameliorating an ocular disease or disorder in a subject in need thereof. The method involves administering to the subject an agent that mobilizes a bone marrow derived stem cell in the subject; inducing heat shock in an ocular tissue; and recruiting the stem cell to the ocular tissue, thereby ameliorating the ocular disease or disorder.

In a related aspect, the invention features a method of ameliorating macular degeneration in a subject in need thereof. The method involves administering to the subject GM-CSF and/or Stem Cell Factor, wherein the administration mobilizes a bone marrow derived stem cell in the subject; inducing heat shock in an ocular tissue by administering a subthreshold laser treatment or pharmacological agent; and recruiting the bone marrow derived stem cell to the ocular tissue, thereby ameliorating the macular degeneration.

In another aspect, the invention features a pharmaceutical composition for stem cell recruitment, the composition containing an effective amount of a small compound selected from the group consisting of geldanamycin, celastrol, 17-allylamino-17-demethoxygeldanamycin, EC102, radicicol, geranylgeranylacetone, paeoniflorin, PU-DZ8, and H-71 formulated in a pharmaceutically acceptable excipient for ocular delivery.

In another aspect, the invention features a pharmaceutical composition for stem cell recruitment in an ocular tissue, the composition containing an expression vector containing a polynucleotide encoding a heat shock polypeptide (e.g., Hsp100, Hsp90, Hsp70, Hsp60, and Hsp40) formulated in a pharmaceutically acceptable excipient for ocular delivery.

In another aspect, the invention features a pharmaceutical composition for stem cell recruitment in an ocular tissue, the composition containing a polypeptide (e.g., Hsp100, Hsp90, Hsp70, Hsp60, or Hsp40) formulated in a pharmaceutically acceptable excipient for ocular delivery.

In yet another aspect, the kit contains an effective amount of an agent (e.g., a polypeptide, a polynucleotide, or a small compound) that induces a heat shock response in an ocular tissue, and instructions for using the kit to increase stem cell recruitment. In various embodiments, the polypeptide is Hsp100, Hsp90, Hsp70, Hsp60, and Hsp40. In yet other embodiments, the polynucleotide encodes Hsp100, Hsp90, Hsp70, Hsp60, and Hsp40. In still other embodiments, the small compound is selected from the group consisting of geldanamycin, celastrol, 17-allylamino-17-demethoxygeldanamycin, EC102, radicicol, geranylgeranylacetone, paeoniflorin, PU-DZ8, and H-71.

In another aspect, the invention features a method of identifying an agent that increases stem cell recruitment in an ocular tissue, the method involving contacting an ocular cell with a test compound; identifying an increase in the expression or activity of a heat shock polypeptide relative to an untreated ocular cell, thereby identifying a compound that increases stem cell recruitment.

In another aspect, the invention features a method of identifying an agent that increases stem cell recruitment in an ocular tissue, the method involving contacting an ocular cell with a test compound; and identifying an increase in the number of stem cells in the tissue.

In various embodiments of any of the above aspects, the subject has an ocular disease or disorder is selected from the group consisting of diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy. In other embodiments of any of the above aspects, the agent increases the expression or biological activity of a heat shock protein selected from the group consisting of Hsp100, Hsp90, Hsp70, Hsp60, and Hsp40. In yet other embodiments, the agent alters the expression or activity of any one or more of the following proteins: SDF-1, VEGF, HIF-1α, crystallin, hypoxia-inducible factor 1-alpha (HIF-1a), and CXCR-4. In still other embodiments of the above aspects, the method increases the expression of an Hsp70 or Hsp90 polypeptide by at least about 10-fold or by at least 40-fold. In yet other embodiments of any of the above aspects, an agent that increases hematopoietic stem cell mobilization (e.g., GM-CSF and/or SCF) is administered to the subject prior to induction of the heat shock response. In still other embodiments of any of the above aspects, the method further involves administering an anti-inflammatory agent or an anti-angiogenic agent. In still other embodiments, the method further involves administering an agent that supports the survival, proliferation, or transdifferentiation of a hematopoietic stem cell. In still other embodiments of any of the above aspects, the method further involves administering all trans-retinoic acid to enhance the transdifferentiation of the stem cell to a retinal pigment epithelial cell. In still other embodiments of any of the above aspects, the stem cell mobilizing agent is granulocyte macrophage colony stimulating factor or stem cell factor. In yet other embodiments of any of the above aspects, the heat shock is induced using a subthreshold laser treatment or using an agent that is a small compound, a polypeptide, or a nucleic acid molecule positioned for expression in a cell. In yet other embodiments of any of the above aspects, the polypeptide is a heat shock polypeptide. In yet other embodiments, the nucleic acid molecule encodes a heat shock polypeptide (e.g., Hsp70, Hsp90) or encodes a therapeutic polypeptide (e.g., an anti-inflammatory polypeptide or modulator of angiogenesis). In still other embodiments of any of the above aspects, the pharmacological agent is selected from the group consisting of geldanamycin, celastrol, 17-allylamino-17-demethoxygeldanamycin, EC102, radicicol, geranylgeranylacetone, paeoniflorin, PU-DZ8, and H-71. In various embodiments of any of the above aspects, the agent is administered by intravitreal or retro-orbital injection. In still other embodiments of any of the above aspects, the administration induces cellular repair of the RPE layer. In still other embodiments of any of the above aspects, the method further involves administering a vector encoding a therapeutic polypeptide. In still other embodiments, the method further involves administering a substantially purified stem cell (e.g., bone marrow derived cell or hematopoietic stem cell to the subject. In yet other embodiments of any of the above aspects, the stem cell is administered locally by intravitreal or retro-orbital infection or systemically.

The invention provides methods of treating various ocular diseases. Other features and advantages of the invention will be apparent from the detailed description, and from the claims.

### DEFINITIONS

By “agent” is meant any small molecule chemical compound, antibody, nucleic acid molecule, or polypeptide, or fragments thereof.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “analog” is meant a structurally related polypeptide or nucleic acid molecule having the function of a reference polypeptide or nucleic acid molecule.

By “biological activity of a heat shock protein” is meant a chaperone activity or stem cell recruiting activity.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term, is open-ended, allowing for the presence of more than that which is recited so long as basic or novel characteristics of that which is recited is not changed by the presence of more than that which is recited, but excludes prior art embodiments.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “labeled nucleic acid or polypeptide” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

By “expression vector” is meant a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “heat shock” is meant any cellular response to thermal stress. Typically, cells respond to heat shock by increasing the transcription or translation of a heat shock polypeptide (e.g., Hsp70 or 90).

By “heat shock polypeptide” is meant any polypeptide expressed in a cell in response to thermal stress. Exemplary heat shock polypeptides include, but are not limited to, Hsp1100, Hsp90, Hsp70, Hsp60, and Hsp40. An exemplary Hsp70 amino acid sequence is provided at GenBank Accession No. AAA02807. Exemplary Hsp90 amino acid sequence is provided at GenBank Accession Nos. P08238, NP_{—}005339, NP_{—}001017963, and P07900.

By “heat shock response activator” is meant a compound that increases the chaperone activity or expression of a heat shock pathway component. Heat shock pathway components include, but are not limited to, Hsp100, Hsp90, Hsp70, Hsp60, Hsp40 and small HSP family members. Agents or treatments that induce heat shock typically increase the expression or activity of at least one of Hsp70 or Hsp90.

By “hematopoietic stem cell” is meant a bone marrow derived cell capable of giving rise to one or more differentiated cells of the hematopoietic lineage.

By “hematopoietic stem cell mobilization” is meant increasing the number of bone marrow derived stem cells available for recruitment to an organ or tissue in need thereof.

By “ocular disease or disorder” is meant a pathology effecting the normal function of the eye.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “polypeptide” is meant any chain of amino acids, regardless of length or post-translational modification.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription. Exemplary promoters include nucleic acid sequences of lengths 100, 250, 300, 400, 500, 750, 900, 1000, 1250, and 1500 nucleotides that are upstream (e.g., immediately upstream) of the translation start site.

By “recruit” is meant attract for incorporation into a tissue.

By “reduces” or “increases” is meant a negative or positive alteration, respectively, of at least 10%, 25%, 50%, 75%, or 100%.

By “regenerating the retina” is meant increasing the number, survival, or proliferation of cells in the retina or retinal pigmented epithelium.

By “repairing retinal pigment epithelium damage” is meant ameliorating retinal pigment epithelium injury, damage, or cell death.

By “stem cell” is meant a progenitor cell capable of giving rise to one or more differentiated cell types.

By “subject” is meant a mammal, including, but not limited to, a human or non-human mammal, such as a bovine, equine, canine, ovine, or feline.

By “subthreshold laser” is meant a laser therapy that induces a lesion that is undetectable or barely detectable in the retina during or following treatment. A lesion is “undetectable” where little or no intraoperative visible tissue reaction is present or where little or no cell death (e.g., less than 10%, 5%, 2.5%, 1% of cells in treated tissue die or apoptose) due to laser treatment.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

By “transdifferentiation” is meant altering the cell, such that it expresses at least one polypeptide characteristically expressed by a cell of a different type.

### BRIEF DESCRIPTION OF THE DRAWINGS

FIG. 1 shows a series of ocular tissue mounts. The dark regions in FIG. 1 represent GFP⁺ cells that have incorporated into the RPE layer in areas that have received laser. Background fluorescence, as determined by the contralateral (unaffected) eye, was removed.

FIG. 2 is a graph quantitating hematopoietic stem cell (HSC) incorporation into the retinal pigment epithelium (RPE).

FIG. 3 is a series of panels showing ocular tissue mounts taken from mice that received suborbital injection of GFP⁺HSC in combination with pharmacologically induced heat shock using geldanamycin derivatives D28 or H71; that received HSP70 polypeptide injection.

### DETAILED DESCRIPTION OF THE INVENTION

The invention generally features compositions and methods that are useful for treating or preventing an ocular disease. The invention is based, at least in part, on the discovery that laser or pharmacological induction of heat shock in the retinal pigment epithelial (RPE) layer and choroid caused hematopoietic stem cells to be recruited to the RPE, where they transdifferentiated into cells expressing markers specific to retinal pigment epithelium cells. Without wishing to be bound by theory, this homing response is due at least in part to activation of the heat shock response.

As reported in more detail below, sub-visible threshold laser stimulation of the retinal pigment epithelium and choroid was used to recruit HSCs to the RPE layer. Adoptive transfer of GFP-labeled HSCs and GFP chimeric animals, were used to demonstrate that HSCs cells can migrate to the RPE layer. SVL induced the expression of heat shock proteins and the subsequent expression of the HSC chemoattractants stromal derived factor (SDF-1) and VEGF. The laser-induced effect could be recapitulated by the intravitreal administration of compounds that chemically induce the heat shock response. Recruited HSCs acquired the morphological characteristics of mature RPE cells and also expressed RPE-specific proteins. Thus, the present invention provides methods for the treatment of subjects having an ocular disease, such as diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy.

### Hematopoietic Stem Cells

Hematopoietic stem cells are bone marrow-derived cells that represent an endogenous source known for their reparative potential as well as for their plasticity. Bone marrow-derived hematopoietic stem cells (HSCs) are able to repair damaged tissues, including heart, liver, brain, muscle and kidney. As reported herein, hematopoietic stem cells can also be used to repair the retina. Sub-threshold laser (STL) stimulation of the retina induced recruitment of HSCs that subsequently transdifferentiated into RPE-like cells. Without wishing to be bound by theory, this process is mediated, at least in part, by molecular interactions that involve chemokines, such as stromal derived growth factor-1 (SDF-1) and chemokine receptors, such as the SDF-1 receptor (CXCR-4) that are activated by subthreshold laser.

Stem cells are recruited to areas of injury to effect the repair of the injured tissue. If desired, the number of hematopoietic stem cells present in the circulation of a subject may be increased prior to, during, or following induction of heat shock. In one embodiment, this increase in hematopoietic stem cell number is accomplished by mobilizing hematopoietic stem cells present in the bone marrow of the subject by administering any one or more of granulocyte-macrophage colony stimulating factor (G-CSF), stem cell factor (SCF), IL-8, SDF-1 (stromal derived factor), interleukin-1 (IL-1), interleukin-3 (IL-3), interleukin-6 (IL-6), interleukin-7 (IL-7), interleukin-8 (IL-8), interleukin-11 (IL-11), interleukin-12 (IL-12), and NIP-1α, stem cell factor (SCF), funs-like tyrosine kinase-3 (flt-3), transforming growth factor-β (TGF-β), an early acting hematopoietic factor, described, for example in WO 91/05795, and thrombopoietin (Tpo), FLK-2 ligand, FLT-2 ligand, Epo, Oncostatin M, and MCSF. SDF-1 is a potent cytokine that induces the recruitment of stem cells. SDF-1 is expressed by RPE cells during stress. Administration of G-CSF and/or SDF-1 will increase the number of HSC in the peripheral blood and will likely enhance subsequent HSC recruitment to the retina and RPE layer. Preferably, hematopoietic stem cells of the invention fail to express or express reduced levels of any one or more of the following markers: Lin⁻, CD2⁻, CD3⁻, CDT7⁻, CD8⁻, CD10⁻, CD14⁻, CD15⁻, CD16⁻, CD19⁻, CD20⁻, CD33⁻, CD38⁻, CD71⁻, HLA-DR⁻, and glycophorin A⁻.

### Ophthalmic Lasers

Ophthalmic lasers are an important tool for the treatment of various retinal disorders where they have typically been used to generate laser-induced photochemical burns. In contrast, the diode 810 nanometer laser is believed to cause less damage to the neurosensory retina because the energy is absorbed by the RPE. The present invention provides methods for using a subvisible laser application to mobilize hematopoietic stem cells and recruit them to the retinal pigment epithelium layer. In the present method, an infrared (810 nm) laser is used in micropulse mode for the treatment of retinal disorders. By using repetitive, brief pulses of laser during a single exposure, it limits the amount of heat conduction and subsequent RPE damage. In one approach, laser administration is controlled to reduce or eliminate photothermal damage. For example, the laser treatment is controlled to reduce or eliminate intraoperative visible tissue reaction (e.g., photocoagulation necrosis) and or late cellular death (apoptosis) In other examples, the threshold of non-lethal thermal injury is controlled such that intraoperative visible tissue reaction is reduced or absent, late cellular death is reduced or absent, and consistent positive HSC recruitment is present. Preferably, the photothermal damage is reduced by at least 10%, 25%, or 30% relative to a patient treated with conventional laser therapy; more preferably, photothermal damage is reduced by at least 50%, 75%, 85%, 95% or 100%. More preferably, the patient's visual acuity is substantially preserved (e.g., is preserved at or near the patient's current level of visual acuity).

Methods of inducing heat shock using a sub-threshold laser include, for example, administering a grid pattern of 40-50 well-spaced 810 nm-laser spots with a diameter of 5 μm, 10 μm, 25 μm, 35 μm, or 50 μm. The power and delivery modalities may be varied to reduce or eliminate photothermal damage. For example, a continuous-wave (cw) delivery mode; a microPulse (mP) delivery mode (e.g., using 20%, 15%, 10% and 5% duty cycle); or a long pulse delivery mode may be used.

In particular embodiments, the present methods feature the use of a sub-threshold laser having a wavelength from at least about 100 nm up to 2000 nm, where the sub-threshold laser energy is at least about 10 mW to 100 mW (e.g., 10, 20, 30, 40, 50, 60, 70, 80, 90 or 100). The laser is applied for a duration of at least 0.001, 0.005, 0.1, 0.2 or 1.0 msec. In other embodiments, 10 mW is administered in a 0.1 msec pulse or 100 mW is administered in a 0.1 msec pulse.

Ocular tissues amenable to laser treatment include the choroid, retinal pigment epithelium, or any other ocular tissue where stem cell repair of tissue damage is desirable. While specific examples described herein relate to the use of lasers to induce heat shock, one skilled in the art appreciates that the invention is not so limited. Virtually any method of energy delivery capable of inducing the heat shock response in at least one cell of an ocular tissue may be used. Such methods include, for example, stimulation using radiation, transpupillary thermography or any other form of energy, such as light energy, in an amount sufficient to stimulate stem cell recruitment may be used. In various embodiments, laser stimulation sufficient to recruit stem cells refers to a light beam, or photons that have a wavelength of from about 100 nm up to 2000 nm. Usually the wavelength is between about 500 nm to about 900 nm.

### Heat Shock Response Activators

Heat shock response activators include agents (e.g., small compound, polypeptide, and nucleic acid molecules) that induce a heat shock response in a cell. Such agents increase, for example, the expression of biological activity of a heat shock protein, such as Hsp100, Hsp90, Hsp70, Hsp60, Hsp40 and small HSP family members. More preferably, the agent increases the expression or biological activity of Hsp90 or Hsp70. Heat shock protein 90 (Hsp90) is a chaperone involved in cell signaling, proliferation and survival, and is essential for the conformational stability and function of a number of proteins. HSP90 modulators are useful in the methods of the invention, such modulators increase the expression or the biological activity of a HSP90. HSP90 modulators include benzoquinone ansamycin antibiotics, such as geldanamycin and 17-allylamino-17-demethoxygeldanamycin (17-AAG), which specifically bind to Hsp90, and alter its function. Other Hsp90 modulators include, but are not limited to, radicicol, novobiocin, and any Hsp90 inhibitor that binds to the Hsp90 ATP/ADP pocket.

Other agents that induce heat shock include, but are not limited to, geldanamycin, (InvivoGen, San Diego, Calif.; Chang et al., J Cell Biochem. 2006 Jan. 1; 97(1):156-65), celastrol, 17-allylamino-17-demethoxygeldanamycin, InvivoGen, San Diego, Calif.), EC102, radicicol (Chang et al., J Cell Biochem. 2006 Jan. 1; 97(1):156-65), geranylgeranylacetone (Eisai, Tokyo, Japan), paeoniflorin (Axxora, San Diego, Calif.), PU-DZ8, and H-71 (H-71 (Santa Cruz Biotechnology, Inc., Santa Cruz, Calif.), as well as analogs or mimetics of any of these compounds. Celastrol, a quinone methide triterpene, activates the human heat shock response. Celastrol and other heat shock response activators are useful for the treatment of ocular disease. Heat shock response activators include, but are not limited to, celastrol, celastrol methyl ester, dihydrocelastrol diacetate, celastrol butyl ester, dihydrocelastrol, and salts or analogs thereof.

### Ocular Disease

The invention may be used for the treatment of ocular diseases that include pre-proliferative retinopathy, diabetic retinopathy, choroidal neovascularization, glaucoma, retinitis pigmentosa, age-related macular degeneration, corneal dystrophies, retinoschises, Stargardt's disease, autosomal dominant druzen, and Best's macular dystrophy.

In particular, the invention provides for the treatment of neovascularization related to proliferative diabetic retinopathy and choroidal neovascularization. Type I diabetes is associated with a high risk for proliferative diabetic retinopathy (Jacobsen, N. et al. 2003 Ugeskr Laeger 165:2953-6). Chronic exposure to the diabetic mileau typically leads to pre-proliferative retinopathy. Pre-proliferative retinopathy is associated with focal areas of ischemia. It is widely accepted that neovascularization is associated with increased expression of pro-angiogenic factors such as vascular endothelial growth factor (VEGF), along with reduced expression of anti-angiogenic factors, such as endostatin and pigment epithelial derived factor, PEDF (Funatsu, H., et al. 2003 Invest Opthalmol V is Sci 44:1042-7; Noma, H., et al. 2002 Arch Opthalmol 120:1075-80; Dawson, D. W. et al. 1999 Science 285:245-8; Spranger, J., et al. 2001 Diabetes 50:2641-5; Holekamp, N. M. et al 2002 Am J Opthalmol 134:220-7; Boehm, B. O., et al. 2003 Horm Metab Res 35:382-6). The change in the balance between pro-angiogenic and anti-angiogenic factors elicits neovascularization and induces capillary leakage (Funatsu, H., et al. 2002 Am J Opthalmol 133:70-7; Caldwell, R. B., et al. 2003 Diabetes Metab Res Rev 19:442-55; Antcliff, R. J. et al. 1999 Semin Opthalmol 14:223-32). After several years, patients having pre-proliferative retinopathy experience retinal pathology characterized by the extensive loss of retinal capillaries and cotton wool spots, followed by the development of new vessels that grow from the retina into the normally avascular vitreous. The fragile new vessels are prone to leakage, causing macular edema and blurry vision. Susceptible to breakage, rupture of these abnormal vessels can result in immediate vision loss.

If permitted to grow, the neovascularization can form blinding fibrovascular membranes and cause the retina to detach. Under presently available treatment protocols, proliferative diabetic retinopathy is treated at the proliferative stage of the condition by placing a grid of laser burns over the retina. This destructive treatment results in substantial vision loss. After a 20 year duration of diabetes, 33% of young adults have received such laser treatments, with an associated decrease in visual acuity and visual angle (Kokkonen, J. et al. 1994 Acta Paediatr 83:273-8; Early Treatment Diabetic Retinopathy Study Research Group 1991 Ophthalmology 98:766-85; Davies, N. 1999 Eye 13 (Pt 4):531-6; Dosso, A. A. et al. 2000 Diabetes Care 23:1855).

Another condition that can be treated with the methods of the invention is choroidal neovascularization. Choroidal neovascularization is responsible for significant loss of vision associated with age-related macular degeneration (AMD), for example. In abnormal choroidal neovascularization, new vessels grow from the choroid into the subretinal space. Retinas at high risk for choroidal neovascularization are identified by the presence of multiple or large soft drusen, reticular drusen, and/or pigmentary changes (Macular Photocoagulation Study Group 1997, Arch Opthalmol 115:741-7; Arnold, J. J. et al. 1995 Retina 15:183-91). VEGF, a hypoxia-regulated protein, is associated with choroidal neovascularization (Frank, R. N., et al. 1996 Am J Opthalmol 122:393-403; Ishibashi, T. et al. 1997 Arch Clin Exp Opthalmol 235:159-67; Kwak, N. et al. 2000 Invest Opthalmol Vis Sci 41:3158-64).

Retinal degeneration is another condition amenable to treatment using the methods of the invention. Retinal degenerative diseases include those diseases characterized by retinal neuron injury or retinal neuron cell death. Retinal neurons include, but are not limited to, photoreceptors and retinal ganglion cells. The retinal degenerative diseases include inherited, acquired, and inflammation-induced retinal degenerative diseases. Inherited retinal degenerative diseases include, for example, all forms of macular degeneration (e.g., dry and exudative age-related macular degeneration), Stargardt's disease, Best's disease, glaucoma, retinitis pigmentosa, and optic nerve degeneration. Acquired retinal degenerative diseases include those associated with cystoid macular edema, retinal detachment, photic damage, ischemic retinopathies due to venous or arterial occlusion or other vascular disorders, retinopathies due to trauma, surgery, or penetrating lesions of the eye, and peripheral vitreoretinopathy. Inflammation-induced retinal degenerative diseases include those associated with viral-, bacterial- and toxin-induced retinal degeneration, and/or uveitis, as well as those that result in optic neuritis.

Other diseases and disorders susceptible to treatment using the methods of the invention include X-linked juvenile retinoschisis, glaucoma, Malattia Leventinese (ML) and Doyne honeycomb retinal dystrophy (DHRD), and corneal dystrophies.

This invention also provides methods for preventing cellular damage in retinal neurons, including damage associated with post-surgical trauma and complications from subsequent exposure to damaging bright light in a protective modality.

### Screening Assays

As discussed herein, compounds that induce heat shock or stem cell recruitment to the retinal pigment epithelium are useful in the methods of the invention. Any number of methods are available for carrying out screening assays to identify such compounds. In one approach, the expression of an HSP polypeptide or nucleic acid molecule is monitored in a cell (e.g., an ocular cell, such as a cell of the choroid or retinal pigment epithelium in vitro or in vivo); the cell is contacted with a candidate compound; and the effect of the compound on HSP polypeptide or nucleic acid molecule expression is assayed using any method known in the art or described herein. A compound that increases the expression of an HSP polypeptide or nucleic acid molecule in the contacted cell relative to a control cell that was not contacted with the compound, is considered useful in the methods of the invention. Alternatively, compounds are screened to identify those that increase stem cell recruitment to the retina. In one embodiment, stem cell recruitment is assayed in a chimeric mouse injected locally or systemically with GFP⁺ expressing stem cells. The presence of GFP⁺ cells is assayed, for example, by examining retinal flat mounts using fluorescence microscopy. Compounds that the number of stem cells recruited to the retina are useful in the methods of the invention. In other embodiments, the survival or differentiation of such cells is assayed using cell specific markers. In a related approach, the screen is carried out in the presence of 11-cis-retinal, 9-cis-retinal, or an analog or derivative thereof. Useful compounds increase the number of stem cells recruited to the retina by at least 10%, 15%, or 20%, or preferably by 25%, 50%, or 75%; or most preferably by at least 100%.

If desired, the efficacy of the identified compound is assayed in an animal model having a ocular disease (e.g., an animal model of retinitis pigmentosa) or having diabetes.

### Test Compounds and Extracts

In general, compounds capable of inducing a heat shock response in a cell or increasing stem cell recruitment to an ocular tissue are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in recruiting stem cells or inducing heat shock should be employed whenever possible.

When a crude extract is found to recruit stem cells or induce heat shock further fractionation of the positive lead extract is necessary to isolate chemical constituents responsible for the observed effect. Thus, the goal of the extraction, fractionation, and purification process is the careful characterization and identification of a chemical entity within the crude extract that induce heat shock or stem cell recruitment. Methods of fractionation and purification of such heterogenous extracts are known in the art. If desired, compounds shown to be useful agents for the treatment of any pathology related to an ocular disease requiring the repair or regeneration of an ocular tissue are chemically modified according to methods known in the art.

### Pharmaceutical Compositions

The present invention features pharmaceutical preparations comprising agents capable of inducing or replicate heat shock (e.g., by increasing the expression or activity of Hsp70 or Hsp90) in an ocular tissue together with pharmaceutically acceptable carriers. Such preparations comprising polypeptide, polynucleotide, or small compounds have both therapeutic and prophylactic applications. Agents useful in the methods described herein include those that increase the expression or biological activity of an Hsp90 polypeptide, or HSP70, or that otherwise induce a heat shock response in an ocular tissue thereby recruiting a stem cell to the tissue. If desired, the compositions of the invention are formulated together with agents that increase the number of hematopoietic stem cells present in the circulation of a subject, for example, by mobilizing hematopoietic stem cells present in the bone marrow of the subject.

Agents that increase the mobilization or recruitment of stem cells include, but are not limited to, antiblastic drugs and G-CSF or GM-CSF, interleukin-1 (IL-1), interleukin-3 (IL-3), interleukin-6 (IL-6), interleukin-7 (IL-7), interleukin-8 (IL-8), interleukin-11 (IL-11), interleukin-12 (IL-12), and NIP-1α, stem cell factor (SCF), fims-like tyrosine kinase-3 (flt-3), transforming growth factor-β (TGF-β), an early acting hematopoietic factor, described, for example in WO 91/05795, and thrombopoietin (Tpo), FLK-2 ligand, FLT-2 ligand, Epo, Oncostatin M, and MCSF.

If desired, compositions of the invention may be formulated together with compounds that enhance the transdifferentiation of a hematopoietic stem cell to an retinal pigment epithelial cell. Such compounds include trans-retinoic acid, 11-cis-retinal or 9-cis-retinal.

Compounds of the invention may be administered as part of a pharmaceutical composition. The compositions should be sterile and contain a therapeutically effective amount of the agents of the invention in a unit of weight or volume suitable for administration to a subject. The compositions and combinations of the invention can be part of a pharmaceutical pack, where each of the compounds is present in individual dosage amounts.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution.

The compounds may be combined, optionally, with a pharmaceutically acceptable excipient. The term “pharmaceutically-acceptable excipient” as used herein means one or more compatible solid or liquid filler, diluents or encapsulating substances that are suitable for administration into a human. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be also included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having an ocular disease or disorder, an effective amount is sufficient to induce heat shock in at least one cell of an ocular tissue; sufficient to attract at least one stem cell to the tissue; or sufficient to stabilize, slow, or reduce a symptom associated with an ocular pathology. Generally, doses of the compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one preferred embodiment, a composition of the invention is administered intraocularly. Other modes of administration include oral, rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Compositions comprising a composition of the invention can be added to a physiological fluid, such as to the intravitreal humor. Oral administration can be preferred for prophylactic treatment because of the convenience to the patient as well as the dosing schedule.

Pharmaceutical compositions of the invention can optionally further contain one or more additional proteins as desired. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian protein which has been introduced according to standard recombinant DNA techniques; or from the human biological fluids (e.g., blood, milk, lymph, urine or the like) or from transgenic animals that contain a gene that expresses a human protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery, system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describeabiocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

### Methods of Ocular Delivery

Compositions of the invention are typically delivered to the eye for treatment of an ocular disease (e.g., diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy). In one embodiment, a composition of the invention is administered through an ocular device suitable for direct implantation into the vitreous of the eye. The compositions of the invention may be provided in sustained release compositions, such as those described in, for example, U.S. Pat. Nos. 5,672,659 and 5,595,760. Such devices are found to provide sustained controlled release of various compositions to treat the eye without risk of detrimental local and systemic side effects. An object of the present ocular method of delivery is to maximize the amount of drug contained in an intraocular device or implant while minimizing its size in order to prolong the duration of the implant. See, e.g., U.S. Pat. Nos. 5,378,475; 6,375,972, and 6,756,058 and U.S. Publications 20050096290 and 200501269448. Such implants may be biodegradable and/or biocompatible implants, or may be non-biodegradable implants. Biodegradable ocular implants are described, for example, in U.S. Patent Publication No. 20050048099. The implants may be permeable or impermeable to the active agent, and may be inserted into a chamber of the eye, such as the anterior or posterior chambers or may be implanted in the schlera, transchoroidal space, or an avascularized region exterior to the vitreous. Alternatively, a contact lens that acts as a depot for compositions of the invention may also be used for drug delivery.

In a preferred embodiment, the implant may be positioned over an avascular region, such as on the sclera, so as to allow for transcleral diffusion of the drug to the desired site of treatment, e.g. the intraocular space and macula of the eye. Minimally invasive transscleral delivery can be used to deliver an effective amount of the active compounds to retina with negligible systemic absorption. Transscleral delivery utilizes the sclera's large and accessible surface area, high degree of hydration which renders it conductive to water-soluble substances, hypocellularity with an attendant paucity of proteolytic enzymes and protein-binding site, and permeability that does not appreciably decline with age. An osmotic pump loaded with active compounds can be implanted in a subject so that the active compounds are transsclerally delivered to retina in a slow-release mode. (Ambati, et al., Invest. Opthalmol. Vis. Sci., 41: 1186-91 (2000)) Furthermore, the site of transcleral diffusion is preferably in proximity to the macula. Examples of implants for delivery of an a composition include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

Examples include, but are not limited to the following: a sustained release drug delivery system comprising an inner reservoir comprising an effective amount of an agent effective in obtaining a desired local or systemic physiological or pharmacological effect, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end; a method for administering a compound of the invention to a segment of an eye, the method comprising the step of implanting a sustained release device to deliver the compound of the invention to the vitreous of the eye or an implantable, sustained release device for administering a compound of the invention to a segment of an eye; a sustained release drug delivery device comprising: a) a drug core comprising a therapeutically effective amount of at least one first agent effective in obtaining a diagnostic effect or effective in obtaining a desired local or systemic physiological or pharmacological effect; b) at least one unitary cup essentially impermeable to, the passage of the agent that surrounds and defines an internal compartment to accept the drug core, the unitary cup comprising an open top end with at least one recessed groove around at least some portion of the open top end of the unitary cup; c) a permeable plug which is permeable to the passage of the agent, the permeable plug is positioned at the open top end of the unitary cup wherein the groove interacts with the permeable plug holding it in position and closing the open top end, the permeable plug allowing passage of the agent out of the drug core, through the permeable plug, and out the open top end of the unitary cup; and d) at least one second agent effective in obtaining a diagnostic effect or effective in obtaining a desired local or systemic physiological or pharmacological effect; or a sustained release drug delivery device comprising: an inner core comprising an effective amount of an agent having a desired solubility and a polymer coating layer, the polymer layer being permeable to the agent, wherein the polymer coating layer completely covers the inner core.

Other approaches for ocular delivery include the use of liposomes to target a compound of the present invention to the eye, and preferably to retinal pigment epithelial cells, choroid cell, and/or Bruch's membrane. For example, the compound may be complexed with liposomes in the manner described above, and this compound/liposome complex injected into patients with an ocular disease, using intravenous injection to direct the compound to the desired ocular tissue or cell. Directly injecting the liposome complex into the proximity of the retinal pigment epithelial cells or Bruch's membrane can also provide for targeting of the complex. In a specific embodiment, the compound is administered via intra-ocular sustained delivery (such as VITRASERT or ENVISION). In a specific embodiment, the compound is delivered by posterior subtenons injection. In another specific embodiment, microemulsion particles containing the compositions of the invention are delivered to ocular tissue to take up lipid from Bruch's membrane, retinal pigment epithelial cells, or both.

Nanoparticles are a colloidal carrier system that has been shown to improve the efficacy of the encapsulated drug by prolonging the serum half-life. Polyalkylcyanoacrylates (PACAs) nanoparticles are a polymer colloidal drug delivery system that is in clinical development, as described by Stella et al., J. Pharm. Sci., 2000. 89: p. 1452-1464; Brigger et al., Int. J. Pharm., 2001. 214: p. 37-42; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly (lactic-acid) (PLA) or poly (lactic-co-glycolic acid) (PLGA) is used. Such polymers are biocompatible, and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the invention include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the invention include PEG-PLGA polymers.

Compositions of the invention may, also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that the best treatment regimens for using compounds of the present invention to treat an ocular disease can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Combination Therapies

Compositions and methods of the invention may be administered in combination with any standard therapy known in the art. If desired, an agent that induces heat shock in an ocular tissue or a subthreshold laser regimen (described herein) is administered together with an agent that promotes the recruitment, survival, proliferation or transdifferentiation of a stem cell (e.g., a hematopoietic stem cell). Such agents include collagens, fibronectins, laminins, integrins, angiogenic factors, anti-inflammatory factors, glycosaminoglycans, vitrogen, antibodies and fragments thereof, functional equivalents of these agents, and combinations thereof.

In other embodiments, an agent that induces heat shock in an ocular tissue or a subthreshold laser regimen (described herein) of the invention is administered in combination with an anti-inflammatory compound that is conventionally administered for the treatment of an ocular disease. Such anti-inflammatory compounds include, but are not limited to, any one or more of steroidal and non-steroidal compounds and examples include: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; or Zomepirac Sodium.

In still other embodiments, an agent that induces heat shock in an ocular tissue or a subthreshold laser regimen of the invention is administered in combination with an agent that increases or decreases angiogenesis in an ocular tissue. Such agents are capable of modulating the expression or activity of an angiogenic factor, such as platelet derived growth factor (PDGF), vascular endothelial growth factor (VEGF), basic fibroblast growth factor (bFGF), bFGF-2, leptins, plasminogen activators (tPA, uPA), angiopoietins, lipoprotein A, transforming growth factor-β, bradykinin, angiogenic oligosaccharides (e.g., hyaluronan, heparan sulphate), thrombospondin, hepatocyte growth factor (also known as scatter factor) and members of the CXC chemokine receptor family.

In other embodiments, agents and methods are administered together with chemotherapeutic agents that enhance bone marrow-derived stem cell mobilization, including cytoxan, cyclophosphamide, VP-16, and cytokines such as GM-CSF, G-CSF or combinations thereof.

Combinations of the invention may be administered concurrently or within a few hours, days, or weeks of one another. In one approach, an agent that induces heat shock in an ocular tissue or a subthreshold laser regimen (described herein) is administered prior to, concurrently with, or following administration of a conventional therapeutic described herein. In some embodiments, it may be desirable to mobilize a bone marrow-derived cell prior to the induction of heat shock, where such mobilization increases the number of stem cells recruited to the ocular tissue. In other embodiments, it may be preferable to administer the agent that mobilizes a bone marrow-derived cell concurrently with or following (e.g., within 1, 2, 3, 5 or 10 hours) of inducing heat shock.

### Methods for Increasing Stem Cell Recruitment to an Ocular Tissue

As reported herein, the induction of heat shock in an ocular tissue effectively recruits stem cells (e.g., hematopoietic stem cells) to that tissue, where they ameliorate an ocular disease or disorder. If desired, substantially purified stem cells (or their precursor or other progenitor cells) are administered to the patient in conjunction with an agent or treatment regimen (e.g., sub-threshold laser treatment) that induces heat shock in an ocular tissue to facilitate the repair of an ocular tissue. Such methods may be used to enhance the repair of an ocular tissue by increasing the recruitment of stem cells to that tissue.

Methods of isolating hematopoietic stem cells are known in the art. In one embodiment, hematopoietic stem cells are isolated from the blood using apheresis. Apheresis for total white cells begins when the total white cell count is about 500-2000 cells/μl and the platelet count is about 50,000/μl. Daily leukapheris samples may be monitored for the presence of CD34⁺ and/or Thy-1⁺ cells to determine the peak of stem cell mobilization and, hence, the optimal time for harvesting peripheral blood stem cells. Various techniques may be employed to separate the cells by initially removing cells of dedicated lineage (“lineage-committed” cells), if desired. Monoclonal antibodies are particularly useful for identifying markers associated with particular cell lineages and/or stages of differentiation. The antibodies may be attached to a solid support to allow for crude separation. The separation techniques employed should maximize the viability of the fraction to be collected.

The use of separation techniques include those based on differences in physical properties (e.g., density gradient centrifugation and counter-flow centrifugal elutriation), cell surface properties (lectin and antibody affinity), and vital staining properties (mitochondria-binding dye rhodamine 123 and DNA-binding dye Hoechst 33342). Other procedures for separation that may be used include magnetic separation, using antibody-coated magnetic beads, affinity chromatography, cytotoxic agents joined to a monoclonal antibody or used in conjunction with a monoclonal antibody, including complement and cytotoxins, and “panning” with antibody attached to a solid matrix or any other convenient technique. Techniques providing accurate separation include flow cytometry (e.g., flow cytometry using a plurality of color channels, low angle and obtuse light scattering detecting channels, impedance channels).

A large proportion of differentiated cells may be removed from a sample using a relatively crude separation, where major cell population lineages of the hematopoietic system, such as lymphocytic and myelomonocytic, are removed, as well as lymphocytic populations, such as megakaryocytic, mast cells, eosinophils and basophils. Usually, at least about 70 to 90 percent of the hematopoietic cells will be removed.

Concomitantly or subsequent to a gross separation providing for positive selection, e.g. using the CD34 marker, a negative selection may be carried out, where antibodies to lineage-specific markers present on dedicated cells are employed. For the most part, these markers include CD2⁻, CD3⁻, CD7⁻, CD8⁻, CD10⁻, CD14⁻, CD15⁻, CD16⁻, CD19⁻, CD20⁻, CD33⁻, CD38⁻, CD71⁻, HLA-DR⁻, and glycophorin A; preferably including at least CD2⁻, CD14⁻, CD15⁻, CD16⁻, CD19⁻ and glycophorin A; and normally including at least CD14⁻ and CD15⁻. As used herein, Lin⁻ refers to a cell population lacking at least one lineage specific marker.

The purified stem cells have low side scatter and low to medium forward scatter profiles by FACS analysis. Cytospin preparations show the enriched stem cells to have a size between mature lymphoid cells and mature granulocytes. Cells may be selected based on light-scatter properties as well as their expression of various cell surface antigens.

Preferably, cells are initially separated by a coarse separation, followed by a fine separation, with positive selection of a marker associated with stem cells and negative selection for markers associated with lineage committed cells. Compositions highly enriched in stem cells may be achieved in this manner.

Purified or partial purified stem cells are then administered to the patient. Administration may be local (e.g., by direct administration to the vitreous humor or to a vessel supplying an ocular tissue of interest) or may be systemic.

### Polynucleotide Therapy to Induce Heat Shock

Polynucleotide therapy featuring a polynucleotide encoding an HSP protein, variant, or fragment thereof or a protein capable of activating heat shock is another therapeutic approach for treating an ocular disease. Alternatively, the polynucleotides encode therapeutic polypeptides that enhance stem cell recruitment, survival, proliferation, or differentiation or otherwise ameliorate a symptom associated with the ocular disease (e.g., reduce inflammation, angiogenesis, or cell death). If desired, a stem cell of the invention is genetically modified to express a bioactive molecule, or heterologous protein or to overexpress an endogenous protein. The cell can carry genetic information required for the long-term survival of the cell, tissue, or organ or for detecting or monitoring the cells. In one example, the cell are genetically modified to express a bioactive molecule that promotes angiogenesis. In another example, the cells are genetically modified to expresses a fluorescent protein marker. Exemplary markers include GFP, EGFP, BFP, CFP, YFP, and RFP. Such polynucleotides can be delivered to cells of a subject having an ocular disease where expression of the recombinant proteins will have a therapeutic effect. For example, nucleic acid molecules that encode therapeutic polypeptides are delivered to stem cells, such as bone marrow-derived stem cells, hematopoietic stem cells, their precursors, or progenitors. In other approaches, nucleic acid molecules are delivered to cells of an ocular tissue, such as the retina, retinal pigment epithelium, or choroid. The nucleic acid molecules must be delivered to the cells of a subject in a form in which they can be taken up so that therapeutically effective levels of the therapeutic polypeptide (e.g., HSP protein, such as HSP 70, HSP 90) or fragment thereof can be produced.

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

If desired, a vector expressing stem cell recruiting factors is administered to an ocular tissue, such as the retinal pigment epithelial layer. SDF-1 (also called PBSF) (Campbell et al. (1998) Science 279(5349):381-4), 6-C-kine (also called Exodus-2), and MIP-3β (also called ELC or Exodus-3) induced adhesion of most circulating lymphocytes, including most CD4⁺ T cells; and MIP-3α (also called LARC or Exodus-1) triggered adhesion of memory, but not naive, CD4⁺T cells. Tangemann et al. (1998) J. Immunol. 161:6330-7 disclose the role of secondary lymphoid-tissue chemokine (SLC), a high endothelial venule (HEV)-associated chemokine, with the homing of lymphocytes to secondary lymphoid organs. Campbell et al. (1998) J. Cell Biol 141(4):1053-9 describe the receptor for SLC as CCR7, and that its ligand, SLC, can trigger rapid integrin-dependent arrest of lymphocytes rolling under physiological shear.

In other approaches, vectors expressing anti-angiogenic polypeptides are administered to reduce neovascularization in an ocular tissue. Such anti-angiogenic polypeptides include, but are not limited to, interferon α, interferon β.

In still other approaches, a vector encoding a polypeptide characteristically expressed in an ocular cell is introduced to a stem cell of the invention. Polypeptides expressed in retinal endothelial cells include the retinal pigment epithelial marker, RPE 65, and endothelial tissue markers, such as RPCA-1. If desired the stem cells constitutively express markers specific for the CNS endothelium (such as P-glycoprotein, GLUT-1, and the transferrin receptor). Stem cells transformed with these vectors preferably exhibit the morphological characteristics and antigen expression characteristics of the retinal pigment epithelial cells (for example, pavement morphology and expression of RET-PE2 and cytokeratins). Examples of retinal specific proteins, including known SAGE tags, are shown in Table 1.

TABLE 1
Retinal-specific/enriched SAGE tags

| | | Retinal |
|---|---|---|
| SAGE tag | Gene | disease |
| TGTGCTGAAC | transferrin | — |
| GTAGGAGCTG | HRG4 | — |
| TACGTGAATT | gamma transducin | — |
| AGAAGGCCTG | ROM1 | ADRP |
| GCCCTGTGCT | recoverin | — |
| CTGGGTAGCA | gamma phosphodiesterase | — |
| GAAAAATAAA | 1. ABCR | Stargardt, |
| | 2. pyruvate dehydrogenase | CRD, ARRP |
| | kinase, isoenzyme | |
| TAACACATTC | 1. hypothetical protein | — |
| | 2. Hs. 151710 | — |
| | 3. sodium calcium exchanger | — |
| CGGGGTAGCA | gamma phosphodiesterase | — |
| GATTTGGATG | Hs. 35493 | — |
| AGAGCGCAGC | crystallin, alpha A | (cataract) |
| GACAATAAAT | MT-protocadherin | — |
| CTCACCACCA | rhodopsin | ADRP, ARRP, |
| | | cSNB |
| CAGATGGTTT | Hs. 21299 | — |
| CACCCTCAGC | arrestin | Oguchi, |
| | | ARRP |
| AGAAAATAAA | 1. secretory carrier | |
| | membrane protein 1 | |
| | 2. phosducin | |
| | 3. Hs. 58033 | — |
| TTGCTTTTAT | 1. chorionic somatomammo- | — |
| | tropin hormone 2 | |
| | 2. Hs. 256635 | — |
| ATCTTTTTAA | 1. MAGUK p55 subfamily | — |
| | member 4 | |
| | 2. cytochrome b-5 | — |
| GTACATTAGT | clusterin-like 1 (retinal) | — |
| GGCCCCAGTT | rhodopsin | ADRP, ARRP, |
| | | cSNB |
| CTAACTGCGA | Hs. 13768 | — |
| CTTTCTCCTT | beta transducin | — |
| ATGGGTCTGG | Hs. 145068 | — |
| AGAGCACAGC | crystallin, alpha A | (cataract) |
| GACCACAAAA | CACNA1F | CSNB |
| ACGTGCGCCA | 1. alpha transducin | CSNB |
| | 2. solute carrier family | |
| | 21) | |
| CTCAGGAATT | beta phosphodiesterase | CSNB |
| TATACCATTT | immunoglobulin superfamily, | — |
| | member 4 | |
| GATGGAGGAC | 1. RNA-binding protein S1 | — |
| | 2. beta channel | ARRP |
| ACTAGCACGC | NRL | ADRP |
| GGAGCCCTCT | NR2E3 | ESCS |
| TAACAAAACC | 1. calcium binding protein | — |
| | 5/3 | |
| | 2. Hs. 57898 | |
| | 3. Hs. 5801 | — |
| ACAATGTTGT | cellular retinoic acid- | — |
| | binding protein 1 | |
| AAGTCTGTTG | Hs. 300880 | — |
| AGGTCTGCCT | red/green opsin | Color |
| | | blindness |
| TACATCATAT | alpha channel | ARRP |
| TGATCACGCC | enolase 2 | — |
| TTAACTGTAC | Hs. 66803 | — |
| ACCCAAGCAT | protein phosphatase, EF | — |
| | hand calcium-binding domain | |
| | 2 (rdgC homolog) | |
| TAGTAGGCAC | RAB4 | — |
| CTGTTACCAG | frizzled-related protein | — |
| TTTCAAAGGG | Hs. 98927 | — |
| TACAGTAGTC | Hs. 239444 | — |
| GTACTTTTAA | Hs. 261526 | — |
| GAAAATGAGA | serine proteinase inhibi- | — |
| | tor, clade B, member 6 | |
| TGGTTGCTGG | Hs. 33792 | — |
| AGGCCGCTAG | X-arrestin | — |
| TAAAATGCAG | 1. neuroD | — |
| | 2. Hs. 271341 | — |
| CAAGGGGTTC | alpha channel | ARRP |
| TATATACACA | 1. IRBP | — |
| | 2. Spindlin | |
| TTTTTGTGTT | 1. Hs. 127019 | — |
| | 2. Hs. 50340 | — |
| GGCTGCAATC | 1. phosphoglycerate mutase | — |
| | 1 | |
| | 2. UDP-Gal: betaGlcNAc beta | — |
| | 1,3-galactosyltransferase | |
| TATGTATCCT | SH3 domain binding glutamic | — |
| | acid-rich protein like 2 | |
| TGCCTGCTAA | Hs. 98881 | — |
| CTTGTTTTGT | 1. retinal homeobox protein | — |
| | 2. Hs. 12799 | |
| TCTACCTATG | 1. glutathione peroxidase 3 | — |
| | 2. calpain 9 | |
| AGCCGGAGGT | dual specificity phospha- | — |
| | tase 6 | |
| ATTTTCAGTT | Hs. 250591 | — |
| TTGGGCAGGC | Hs. 213731 | — |
| AACTTCAAGG | Hs. 154131 | — |
| TGGTAAATGT | guanylate cyclase activator | — |
| | 1C | |
| GGGGACCCTT | HT017 | — |
| CTCTTCTGGA | guanylate cyclase activator | — |
| | 1A | |
| GGAAGGAAAA | 1. ATPase, H+ transporting, | — |
| | lysosomal, member D | |
| | 2. Hs. 43112 | |
| TCCAAACTAA | gamma PDE | — |
| GGGTGGGGGA | 1. vesicle-associated | — |
| | membrane protein 2 | |
| | 2. Hs. 220656 | |
| | 3. Hs. 340046 | |
| TGCTTCTTCT | NDRG family, member 4 | — |
| TGTCAGTCTT | Hs. 310689 | — |
| AAGTTTGGCC | transferrin | — |
| TCCCTGGTGC | 1. synaptogyrin 1 | — |
| | 2. Hs 279307 | |
| TTTTATTGCA | neurobeachin | — |
| ATACTCATTG | Hs. 122245 | — |
| AGAGACCCTC | Hs. 113689 | — |

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an HSP protein, variant, or a fragment thereof, can be cloned into a retroviral vector and expression can be driven from its endogenous promoter, from the retroviral long terminal repeat, or from a promoter specific for a tissue or cell of interest (e.g., an ocular tissue, such as the choroid or the retinal pigment epithelial layer). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an HSP polynucleotide into the eye.

Non-viral approaches can also be employed for the introduction of a therapeutic to a cell of a patient (e.g., an ocular cell or tissue). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTR) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified stem cell and/or retinal cells. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector preferably includes a selection gene, for example, a neomycin resistance gene, for facilitating selection of stem cells that have been transfected or transduced with the expression vector.

If desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant HSP protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

The ocular route is the preferred route of inoculation of drug induced homing of stem cell and/or vector to ocular tissues. However, this designation as the preferred inoculation route is not meant to preclude any other route of administration. Preferred routes of inoculation of the vector are via intravitreal or subretinal routes. The ocular route includes but is not limited to subconjunctival injection, surface drops, a slow-release device such as a collagen shield, a hydrogel contact lens or an ALZA “Ocusert.” Subconjunctival vaccination is done using proparacaine for anesthesia prior to the injection of 0.2-0.5 ml of vector, in a dose of 10-1000 .μg/inoculation, given in an insulin syringe and a small gauge needle. The injection is given in the lower cul-de-sac ensuring that the vaccine material remains subconjunctival and does not leak out.

The surface drops inoculation involves placing the vector with or without adjuvant and/or stem cell trafficking drug in the conjunctival cul-de-sac and then rubbing the eye gently for 30 seconds while held closed. The procedure can be repeated two or three times a day for five days to prolong the exposure, all of which comprise a single vaccination. For better retention, the tear drainage ducts may be temporarily blocked using collagen or other devices. Alternatively, the vector and/or naked DNA may be encapsulated in a microcapsule and then implanted into the eye to facilitate continuous release.

### Kits

The invention provides kits for the treatment or prevention of an ocular disease, disorder, or symptoms thereof. In one embodiment, the kit includes a pharmaceutical pack comprising an effective amount of a Hsp90 chaperone modulator (e.g., Geldanamycin) or a heat shock response activator (e.g., Celastrol). Preferably, the compositions are present in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing an ocular disease or disorder, such as diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy. The instructions will generally include information about the use of the compounds for the treatment or prevention of an ocular disease or disorder. In other embodiments, the instructions include at least one of the following: description of the compound or combination of compounds; dosage schedule and administration for treatment of an ocular disorder or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1

### Recruitment of Stem Cells by Laser

Chimeric mice were constructed with GFP⁺ stems cells that were transplanted into mice that had undergone near lethal irradiation. Chimeric mice were made with GFP-expressing hematopoietic stem cells from GFP homozygous transgenic donors. These cells were transplanted into recipients that had undergone near lethal irradiation. These gfp chimeric mice (C57B16.gfp) were used in all subsequent laser studies. Using the 810 nm diode laser with a spot diameter of 75 μm, various levels of energy were delivered to the retina including energy of 5 mJ (50 mW, 0.1 msec) that did not produce visible laser tissue reaction in the retina (laser irradiance 1130 W/cm²). This was considered sub-visible threshold. Three weeks post-laser the animals were euthanized and the eyes were harvested. Eye cups were prepared and the neurosensory retina was removed.

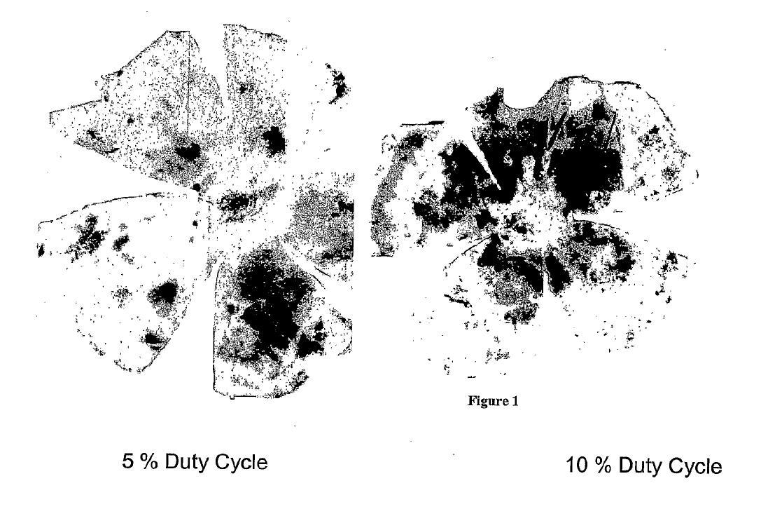

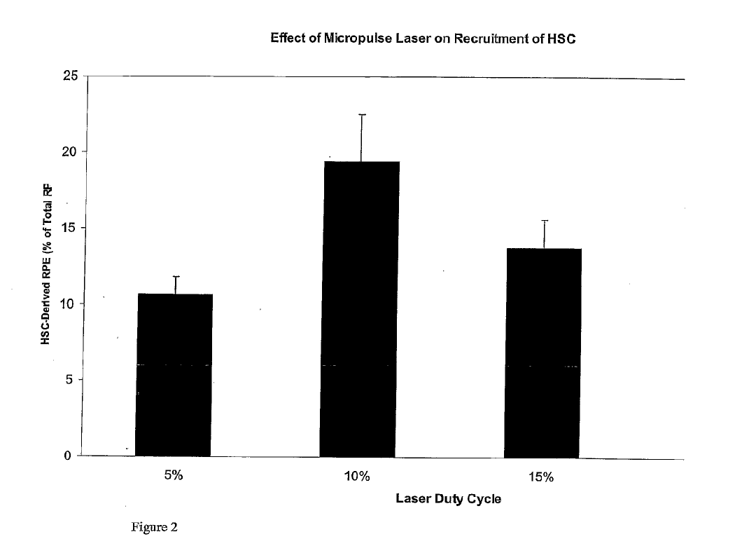

Eyes receiving subthreshold laser energy demonstrated robust recruitment of hematopoietic stem cells (HSC) to the retinal pigment epithelial layer in a diffuse pattern. These changes occurred within a 2 week period. Sub-threshold laser induced adult stem cells to migrate to and repair the retinal pigmented epithelium as shown in FIGS. 1 and 2. FIG. 1 demonstrates the striking duty-cycle dependent localization of gfp⁺ cells at the level of the RPE. The dark regions in FIG. 1 represent GFP⁺ cells that have incorporated into the RPE layer in areas that have received laser. Background fluorescence, as determined by the contralateral (unaffected) eyes was removed from this image. FIG. 2 is a graph quantitating human stem cell (HSC) incorporation into RPE.

### Example 2

### HSC's Recruited to the Retina Express a Retinal Specific Marker

By confocal immunofluorescence microscopy, it was also shown that GFP positive cells co-localized with RPE65—a protein specific for the RPE, suggesting that the recruited hematopoietic stem cells have acquired RPE characteristics. These eyes also demonstrated diffuse endothelial cell recruitment as well. Eyes receiving high energy laser with noticeable retinal opacification (150 mW, 0.1 sec) showed focal recruitment of GFP⁺ HSC to the scar region along with endothelial cells. Sub-threshold laser induced HSC to migrate to and incorporate into the RPE. The degree of incorporation correlates with laser duty cycle. 15% duty cycle resulted in the greatest degree of HSC incorporation.

### Example 3

### Subvisible Threshold Laser Increased hsp70, hsp90, and Induction of the Heat Shock Response Resulted in the Release of SDF-1 and VEGF

Ophthalmic lasers are an important tool for the treatment of various retinal disorders. In most instances, the effect has been attributed to visible changes in the retina (i.e., laser-induced photochemical burns). The diode 810 nanometer laser is believed to cause less damage to the neurosensory retina because the laser energy is absorbed by the RPE. FIG. 2 demonstrated the striking duty-cycle dependent localization of GFP⁺ cells at the level of the RPE. There is a maximal response at 10% duty-cycle. This was separately confirmed using adoptive transfer methods, a technique that closely resembles cellular therapy. Adoptive transfer involves the systemic administration of HSCs and results in the rapid homing of these cells to areas producing chemoattractants.

Micropulse lasering has been developed clinically to minimize photodestructive damage to the retina. The infrared (810 nm) laser used in micropulse mode is a relatively new modality for potential treatment of retinal disorders. Using repetitive, brief pulses of laser during a single exposure limits the amount of heat conduction and subsequent RPE damage. It has recently gained clinical acceptance for this reason. The heat shock response is considered to be a cytoprotective response based on the ability of the ensemble of heat shock proteins to limit protein misfolding.

To determine whether the use of micropulse lasering acted through thermal effects that induce the heat shock response and/or induction of cytokine and growth factors that attract HSCs to the eye, an infrared laser with variable duty cycle was used to examine the time course of mRNA expression of hsp70, hsp90 and crystallins in both the neurosensory retina and the posterior cup which contains the RPE and choroid complex. A peak increase in hsp70 was observed two hours post-laser in the neural retina and four hours post-laser in the posterior eye cup. mRNA for hsp90 dramatically peaked in both the neural retina and the posterior eye cup at two hours. Laser-induced expression of SDF-1 and its receptor CXCR-4 in the posterior eye cup was also observed. Examination at two hours post-laser suggested that a brief laser treatment effected the RPE cell's transcriptional machinery and reprogrammed the RPE cell to produce a series of factors that are capable of recruiting HSCs to the retina. By 4 hours post-laser, an increase in expression of SDF-1 and CXCR-4 was observed. Since SDF-1 and VEGF are known to be responsive to hypoxia, the effect of lasering the retina on HIF-1α mRNA levels was examined. mRNA for HIF-1α was reduced at 2 hours and increased at 4 hours in the posterior eye cup.

These studies support the autocrine and paracrine regulation of RPE by SDF-1 and VEGF. Without wishing to be bound by theory, it is likely that subvisible laser primes the extracellular environment of the RPE—photoreceptor layers and creates a receptive environment for recruiting HSCs. Growing evidence indicates that extracellular hsp70 is a neuroprotective agent. Without wishing to be bound by theory, hsp70 may be acting as a neuroprotective a factor in the retina by facilitating the recruitment of HSCs to the retina to provide for the repair of the RPE. The heat shock proteins likely function in the recruitment of HSC to the retina. This may be accomplished in vivo by the recruitment of HSCs following local production of chemoattractant proteins.

### Example 4

### Hsp70 mRNA Levels Increased Following Heat Shock of Primary RPE Cultures

To determine if the RPE could be a source of the observed in vivo cytokine response, cultures of human primary RPE and ARPE19 (an immortalized RPE cell) were heat shocked. At two hours post heat-shock, suppression of the mRNAs of the heat shock proteins, HIF-1α as well as SDF-1 and VEGF was observed. In RPE cultures, a dramatic forty-fold increase in hsp90 mRNA levels was observed. The in vitro hsp90 results paralleled results in vivo. Strikingly, a fifty-fold increase in hsp70 mRNA levels was observed in the primary RPE cultures, which indicated that resident RPE cells released this putative neuroprotective agent. Based on these in vivo data, and without wishing to be bound by theory, it is likely that RPE cells are the source of chemotactic factors that facilitated HSC recruitment to the retina. This does not preclude the possibility that other cell types participated in the recruitment response.

In sum, these results indicated for the first time that HSCs can be locally recruited to the retina, including the RPE layer by either laser or pharmacological induction. This was achieved with SVL induction of the heat shock response. The laser-induced heat shock response was temporally associated with the release of HIF-1α and then the HSC chemoattractants SDF-1 and VEGF. The present results produced no clinically visible laser burn or scar. This lack of damage distinguishes the present methods from methods that induce visible retinodestructive lasering.

### Example 5

### Chemically Induced Heat Shock Recruits HSC to the RPE

These observations were extended by chemically inducing the heat shock response in primary human RPE cells. Four hours post-laser exposure, there was an exuberant increase in hsp70 levels, and a moderate increase in hsp90, hsp32 and crystallin mRNA levels. SDF-1 expression was also observed. The time course for this expression mirrored that seen during classic heat shock induction.

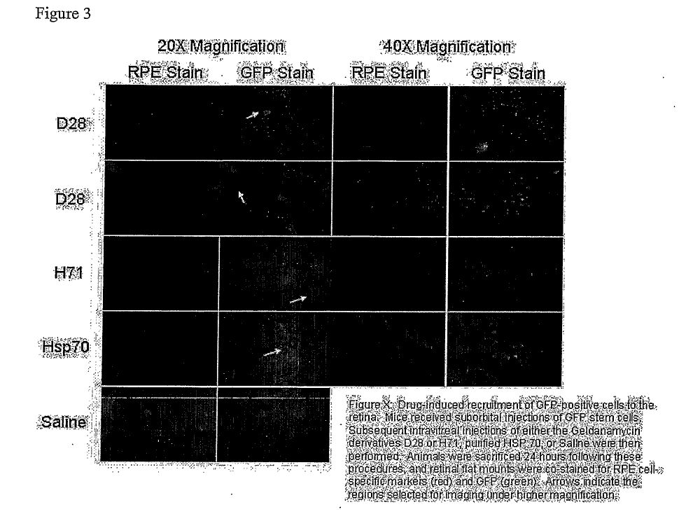

In addition, chemically induced heat shock recruited HSC to the RPE as shown in FIG. 3. The pharmacological induction with small molecule inducers of the heat shock response was induced by the intravitreal injection of geldanamycin or by separately exposing RPE cells resulted in the identical induction of SDF-1 and VEGF. These experiments provide conclusive evidence that heat shock induction, by either laser or pharmacological induction, directly results in the production HIF-1α and the critical HSC chemokines, SDF-1 and VEGF. Further, they suggest pharmacological manipulation effectively leads to HSC recruitment to the RPE layer and differentiation.

Clinical studies have shown that elderly patients have reduced levels of HSCs in their circulation. These cells can be mobilized from the bone marrow to enter the systemic circulation for recruitment to the retina. Currently, the common dry form of ARMD has no effective treatment. The present invention provides laser and pharmacological methods for the treatment of ARMD. The present methods further provide hematopoietic stem cell therapy for ARMD. The present studies suggest that age-related repair defects may contribute to the development of ARMD and further indicate that ARMD may be treated by enhancing repair function using a combination of laser and pharmacological approaches. In particular, the invention provides methods for priming ARMD patients with an agent that mobilizes HSCs, such as GM-CSF, followed by sub-visible laser or intravitreal injection of compounds that can induce the heat shock response would initiate cellular repair of the RPE layer.

The results described above were obtained using the following methods and materials.

### Electroretinography (ERG)

Retinal function of treated and untreated eyes is evaluated by ERG (a non-invasive technique used to determine photoreceptor function) on a periodic (e.g., monthly) basis to determine the effect of laser or pharmacological agent therapy. Electroretinography is a non-invasive technique in which the corneal electrical response to light is measured in anesthetized animals. Mice are anesthetized with intraperitoneal injections of a mix of 80-100 mg/kg ketamine and 5-10 mg/kg xylazine for anesthesia (Phoenix Pharmaceuticals, St. Joseph, Mo.). The mouse corneas are anesthetized with a drop of 0.5% proparacaine HCl (Akorn, Buffalo Grove, Ill.), and dilated with a drop of 2.5% phenylephrine HCl (Akorn). Measurement electrodes tipped with gold wire loops are placed upon both corneas with a drop of 2.5% hypromellose (Akorn) to maintain electrode contact and corneal hydration. A reference electrode is placed subcutaneously in the center of the lower scalp of the mouse, and a ground electrode is placed subcutaneously in the hind leg. The mouse rested on a homemade sliding platform that keeps the animal at a constant temperature of 37° C. The animal is positioned so that its entire head rested inside of the Ganzfeld (full-field) illumination dome of a UTAS-E 2000 Visual Electrodiagnostic System (LKC Technologies, Inc., Gaithersburg, Md.). Full-field scotopic ERGs are measured by 10 msec flashes at an intensity of 0.9 and 1.9 log cd m-2 at 1 minute intervals.

Responses are amplified at a gain of 4,000, filtered between 0.3 to 500 Hz and digitized at a rate of 2,000 Hz on two channels. Five responses are averaged at each intensity. The wave traces analyzed using UTAS-E 2000 software package (LKC Technologies, Inc.). A-waves are measured from the baseline to the peak in the cornea-negative direction; b-waves are measured from the cornea-negative peak to the major cornea-positive peak.

The animals receive triple antibiotic ointment (Vetropolycin) in their eyes to maintain moisture following the procedure, and are allowed to regain consciousness on a 37 degree warming tray before they are returned to the vivarium. Animals receiving Ketamine/Xylazine anesthesia will also receive 0.01-0.02 ml/g of body weight of warm LRS SQ.

### Funduscopy

Retinal examination of treated and untreated eyes is evaluated by funduscopic examination. Funduscopy is a non-invasive technique in which retinal photographs are taken of anesthetized animals. Mice are anesthetized, and their corneas are anesthetized and dilated as described above for ERG analysis. Fundus photography is performed with a specialized camera and lens, a Kowa Genesis hand held fundus camera (Kowa Company, Ltd., Tokyo, Japan) focused through a Volk Super 66 Stereo Fundus Lens (Keeler, Berkshire, England). Two pictures of each eye are generally taken to ensure a properly focused image. The animals receive triple antibiotic ointment (Vetropolycin) in their eyes to maintain moisture following the procedure, and are allowed to regain consciousness on a 37° C. warming tray before they are returned to the vivarium. Animals receiving Ketamine/Xylazine anesthesia will also receive 0.01-0.02 ml/g of body weight of warm LRS SQ.

### Laser Treatment

The pre-surgical preparation of the animals involves making sure that they are physically active and able to undergo anesthesia. The mice need to be without any evidence of ocular discharge or evidence of a cataract so as to make it feasible to visualize the retina to perform laser burn treatment. Prior to the laser treatment the animals will be anesthetized with intraperitoneal injection of a mix of 80-100 mg/kg ketamine and 5-10 mg/kg xylazine. No corneal edema or cataract formation is attributed to the use of these anesthetics. The level of anesthesia is monitored by a footpad pinch and breathing rate. A lack of the reflex-response to the footpad pinch indicates that the animal is properly anesthetized. No ocular ointment is applied before or during the laser treatment because this would prevent effective laser treatment. Some antibiotic ointment is applied to protect the untreated eye. If no response is demonstrated after footpad pinch then the laser treatment will proceed.

Pain, distress or discomfort is suggested by movement of the animal during the time required for laser treatment. If the animal shows increased movement just prior to or during laser surgery then anesthesia is supplemented by exposing the animal to isoflurane for 10 seconds. Approximately 1 mL isoflurane is soaked onto a crumpled Kimwipe placed in the bottom of a 50 mL plastic centrifuge tube and the tube is capped. If necessary the open end of this tube can be held briefly near the animal's nose. This procedure is performed in a fume hood. After this the animals' breathing rate will continue to be monitored and the animal's pain response will be monitored by footpad pinch. If no movement is demonstrated after footpad pinch, then the laser surgery will proceed. The laser treatment takes approximately 30 seconds per mouse. An intraperitoneal injection of yohimbine (2 mg/kg body weight) is used to reverse the effect of the ketamine/xylaxine. This will reduce the amount of time that the eyes are at risk due to the loss of the blink reflex under anesthesia. No abnormal behavior is expected following surgery.

Pain, distress and discomfort can occur after laser treatment. The literature indicates that human recovery from laser treatment is helped by application of ketorolac to the cornea at the end of the procedure (Kosrirukvongs et al, Topical ketorolac tromethamine in the reduction of adverse effects of laser in situ keratomileusist, J. Med Assoc That, 2001; 84:804-810 and Price, et al, Pain reduction after laser in situ keratomileusis with ketorolac tromethamine ophthalmic solution 0.5%: a randomized, double-masked, placebo-controlled trail, J. Refeact Surg, 2002:18:140-144). Therefore, after laser treatment drops of a solution of ketorolac (0.5% OP) will be applied to the eyes of the mice for 48 hours following treatment, and longer if needed. The commercial name of this solution is Acular PF Solution. This duration of treatment with Acular PF has no adverse effects on the mice and has no effect on neovascularizaion as evidenced by analysis of the vehicle injected animals. The animals will be maintained in their cages and held at room temperature (18-26° C.), or on a 37° C. warming tray and visually monitored continuously until they show signs of recovery from anesthesia. Full recovery from anesthesia occurs only when the animal is fully alert and ambulatory in the cage.

### Bone Marrow Harvesting:

Donor mice were humanely euthanized prior to bone marrow harvesting, as this procedure is not a survival surgery. The animals were euthanized by injection with an overdose of ketamine (Xylazine 60 mg/Kg, Ketamine 30 mg/Kg) administered IP. After deep pain loss was achieved, as determined by failure to respond to toe/footpad pinch, a cervical dislocation was performed to confirm death.

To harvest mouse bone marrow derived stem cells, femurs were dissected aseptically from 4-8-week-old transgenic mice expressing enhanced green fluorescence protein (GFP). Both ends of the femurs were cut, and the marrow was extruded with 5 mL of Dulbecco's modified Eagle's medium (DMEM; Nissui Co., Tokyo, Japan) containing 10% fetal bovine serum (FBS), 2 mM L-glutamine, 100 units/mL penicillin G, and 10% heparin, using a 2.5-mL syringe and a 21-gauge needle. FACS analysis was used to confirm the proper BMSC phenotype.

### Intraperitoneal Injections:

The procedure for intraperitoneal injection is as follows: The subjected is picked up by the scruff of its neck as close to the ears as possible. The tail of the mouse will be held by little fingers of the same hand. Using a sterile hypodermic needle of 26 gauge, the skin and abdominal muscle will be pierced and the contents of the needle is injected into the peritoneal cavity with cautions to avoid the diaphragm and other internal organs.

### Intravitreal Injections

Mice are anesthetized as described above. Proparacaine hydrochloride 0.5% (Alcon Laboratories, Inc., Fort Worth, Tex.) is used for additional topical anesthesia. Phenylephrine HCl and/or Atropine Sulfate is applied to the ocular surface before injection in order to achieve dilation of the iris to minimize damage related to the procedure, and triple antibiotic ophthalmic ointment (Vetropolycin) is applied after injection, to prevent infection. A 30-gauge needle is used to make a punch incision 0.5 mm posterior to the temporal limbus, and the microinjector needle is inserted through the incision, approximately 1.5 mm deep, angled toward the optic nerve until the tip of needle is visualized in the center of the vitreous. All injections are visualized with the aid of a dissecting microscope, Nikon SM2800 (Nikon, Melville, N.Y.) with illumination provided by a fiber optic light source from Southern Micro Instruments 150 Watt fiber optic light source with Schott Fostec fiber optic arms (Southern Micron Instruments, Marietta, Ga.) Up to 2 microliters of drug suspension is then slowly introduced into the vitreous, and the needle is carefully withdrawn.

Following this procedure, the mice are allowed to recover in clean cages on a warming tray. Animals are observed until they are fully recovered from the procedure, after which time they are returned to the vivarium.

### Retro-Orbital Injections

For local delivery of stem cells retro-orbital injections work very well for transplants of stem cells as an alternative to tail vein intravenous injections. Mice are anesthetized with either ketamine/xylazine as described above, or through the use of isoflurane administered via a precision vaporizer. The fur above the eye is gently retracted, similar to a bleeding procedure. Injections are performed using a 1 cc syringe and a 27 or 30 g needle, with the bevel facing outward and inserted at a 45° angle into the center of the area of the retro-orbital sinus. The tip of the needle is carefully advanced to penetrate the retro-orbital sinus, with care taken to make sure the needle is approximately mid-sinus. Up to 200 μl of cell suspension is slowly injected. The cell suspension is filtered prior to injection so that it contains no clumps. After injection, the needle is carefully removed, keeping the bevel outward to protect the mouse's eye from being scratched.

For multiple injections, at least 2 days are allowed to elapse between injections. Eyes are alternated for subsequent injections, with a maximum of 2 injections per eye per mouse. Proparacaine topical anesthetic is administered one minute prior to injection

Retro-orbital injection is significantly less traumatic than eye bleeding at the same site. The site is inspected or observed post-procedure to ensure that there is no trauma to the eye. After each injection, and as soon as any bleeding has totally stopped, ophthalmic antibiotic ointment will be applied and spread evenly in the eye to prevent infection. After the procedure, the animal is monitored for pain signs (blepharospasm, squinting) within the next 24-48 hours and given analgesics as needed.

Following anesthesia, the animal(s) are observed until they become conscious and ambulatory, after which they will be returned in their cage(s) to the cage racks.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for ameliorating an ocular disease in a subject, the method comprising
(a) inducing heat shock in at least one cell of an ocular tissue; and
(b) recruiting a stem cell to the ocular tissue, thereby ameliorating the ocular disorder.

2. The method of claim 1, wherein the heat shock is induced in the ocular tissue using sub-visible threshold laser (SVL) stimulation.

3. The method of claim 1, wherein the heat shock is induced using a small compound selected from the group consisting of geldanamycin, celastrol, 17-allylamino-17-demethoxygeldanamycin, EC102, radicicol, geranylgeranylacetone, paeoniflorin, PU-DZ8, and H-71.

4. The method of claim 1, wherein the heat shock is induced using a polypeptide selected from the group consisting of Hsp100, Hsp90, Hsp70, Hsp60, and Hsp40.

5. The method of claim 1, wherein heat shock is induced using an expression vector comprising a polynucleotide encoding a heat shock polypeptide.

6. The method of any one of claims 1-4, wherein the method increases the expression or activity of a heat shock protein selected from the group consisting of Hsp100, Hsp90, Hsp70, Hsp60, and Hsp40.

7. The method of claim 4 or 5, wherein the heat shock polypeptide is Hsp70 or Hsp90.

8. The method of claim 1, wherein the stem cell is a hematopoietic stem cell.

9. The method of claim 1, wherein the method reduces at least one symptom of the ocular disease or disorder.

10. The method of claim 1, wherein the ocular disease or disorder is selected from the group consisting of diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy.

11. A method of recruiting a stem cell to an ocular tissue of a subject in need thereof, the method comprising stimulating the ocular tissue with a sub-threshold laser, wherein the level of stimulation is sufficient to recruit at least one stem cell to the tissue.

12. The method of claim 11, wherein a 10% or 15% duty cycle is used.

13. The method of claim 11, wherein the sub-threshold laser has a wavelength from at least about 100 nm to 2000 nm.

14. The method of claim 11, wherein the sub-threshold laser energy is from about 5 mW to 200 mW.

15. The method of claim 11, wherein the sub-threshold laser energy is from 10 mW to 100 mW.

16. The method of claim 11, wherein the laser is administered in a micropulse.

17. The method of claim 11, wherein the duration of the micropulse is from about 0.001 msec to 1.0 msec.

18. The method of claim 11, wherein the duration of the micropulse is 0.1 msec.

19. The method of claim 10, wherein the sub-threshold laser energy is between 10 mW to 100 mW and is administered in a 0.1 msec pulse.

20. The method of claim 10, wherein the stimulation increases the expression or biological activity of a heat shock protein selected from the group consisting of Hsp100, Hsp90, Hsp70, Hsp60, and Hsp40.

21. The method of claim 10, wherein the stimulation alters the expression or activity of a protein selected from the group consisting of SDF-1, VEGF, HIF-1α, crystallin, hypoxia-inducible factor 1-alpha (HIF-1a), and CXCR-4.

22. The method of claim 10, wherein the method increases the expression of an Hsp70 or Hsp90 polypeptide by at least 10-fold.

23. The method of claim 10, wherein the method increases the expression or activity of an Hsp70 or Hsp90 polypeptide by at least 40-fold.

24. A method of recruiting a stem cell to an ocular tissue of a subject in need thereof, the method comprising
(a) administering an agent to a subject in an amount sufficient to induce heat shock in an ocular tissue; and
(b) recruiting a stem cell to the ocular tissue.

25. A method of ameliorating an ocular disease or disorder in a subject in need thereof, the method comprising
(a) administering an agent to a subject in an amount sufficient to induce heat shock in an ocular tissue; and
(b) recruiting a stem cell to the ocular tissue, thereby ameliorating the ocular disease or disorder.

26. The method of claim 25, wherein the ocular disease or disorder is selected from the group consisting of diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy.

27. A method of regenerating the retina in a subject in need thereof, the method comprising
(a) administering an agent to a subject in an amount sufficient to induce heat shock in an ocular tissue; and
(b) recruiting a stem cell to the ocular tissue, thereby regenerating the retina.

28. A method of repairing retinal pigment epithelium damage in a subject in need thereof, the method comprising
(a) administering an agent to a subject in an amount sufficient to induce heat shock in an ocular tissue; and
(b) recruiting a stem cell to the ocular tissue, thereby repairing the retinal pigment epithelium.

29. The method of any one of claims 22-28, wherein the heat shock is induced using a small compound.

30. The method of claim 29, wherein the small compound is selected from the group consisting of geldanamycin, celastrol, 17-allylamino-17-demethoxygeldanamycin, EC102, radicicol, geranylgeranylacetone, paeoniflorin, PU-DZ8, and H-71.

31. The method of any one of claims 22-28, wherein the heat shock is induced using a polypeptide.

32. The method of any one of claims 22-28, wherein heat shock is induced using an expression vector comprising a polynucleotide encoding a heat shock polypeptide.

33. The method of claim 30 or 31, wherein the heat shock polypeptide is selected from the group consisting of Hsp100, Hsp90, Hsp70, Hsp60, and Hsp40.

34. The method of any one of claims 22-28, wherein the agent increases the expression or biological activity of a heat shock protein selected from the group consisting of Hsp100, Hsp90, Hsp70, Hsp60, and Hsp40.

35. The method of any one of claims 22-28, wherein the agent alters the expression or activity of a protein selected from the group consisting of SDF-1, VEGF, HIF-1α, crystallin, hypoxia-inducible factor 1-alpha (HIF-1a), and CXCR-4,

36. The method of any one of claims 22-28, wherein the method increases the expression of an Hsp70 or Hsp90 polypeptide by at least 10-fold.

37. The method of any one of claims 22-28, wherein the method increases the expression or activity of an Hsp70 or Hsp90 polypeptide by at least 40-fold.

38. The method of any one of claims 1-28, wherein an agent that increases hematopoietic stem cell mobilization is administered to the subject prior to induction of the heat shock response.

39. The method of any one of claims 1-28, wherein the method further comprises administering an anti-inflammatory agent or an anti-angiogenic agent.

40. The method of any one of claims 1-28, wherein the method further comprises administering an agent that supports the survival, proliferation, or transdifferentiation of a hematopoietic stem cell.

41. The method of any one of claim 1-28, wherein the subject has an ocular disease or disorder selected from the group consisting of diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy.

42. The method of any one of claims 1-28, wherein the method further comprises administering all trans-retinoic acid to enhance the transdifferentiation of the stem cell to a retinal pigment epithelial cell.

43. A method of ameliorating an ocular disease or disorder in a subject in need thereof, the method comprising
(a) administering to the subject an agent that mobilizes a bone marrow derived stem cell in the subject;
(b) inducing heat shock in an ocular tissue; and
(c) recruiting the stem cell to the ocular tissue, thereby ameliorating the ocular disease or disorder.

44. The method of claim 43, wherein the agent is granulocyte macrophage colony stimulating factor or stem cell factor.

45. The method of claim 43, wherein the heat shock is induced using a subthreshold laser treatment.

46. The method of claim 43, wherein the heat shock is induced using an agent that is a small compound, polypeptide, or nucleic acid molecule.

47. The method of claim 45, wherein the small compound is selected from the group consisting of geldanamycin, celastrol, 17-allylamino-17-demethoxygeldanamycin, EC 102, radicicol, geranylgeranylacetone, paeoniflorin, PU-DZ8, and H-71.

48. The method of claim 43, wherein the ocular disease or disorder is selected from the group consisting of diabetic retinopathy, choroidal neovascularization, glaucoma retinitis pigmentosa, age-related macular degeneration, glaucoma, corneal dystrophies, retinoschises, Stargardt's disease, autosomal dominant druzen, and Best's macular dystrophy, cystoid macular edema, retinal detachment, photic damage, ischemic retinopathies, inflammation-induced retinal degenerative disease, X-linked juvenile retinoschisis, glaucoma, Malattia Leventinese (ML) and Doyne honeycomb retinal dystrophy.

49. A method of ameliorating macular degeneration in a subject in need thereof, the method comprising
(a) administering to the subject GM-CSF and/or Stem Cell Factor, wherein the administration mobilizes a bone marrow derived stem cell in the subject;
(b) inducing heat shock in an ocular tissue by administering a subthreshold laser treatment or agent; and
(c) recruiting the bone marrow derived stem cell to the ocular tissue, thereby ameliorating the macular degeneration.

50. The method of claim 49, wherein the agent is selected from the group consisting of geldanamycin, celastrol, 17-allylamino-17-demethoxygeldanamycin, EC102, radicicol, geranylgeranylacetone, paeoniflorin, PU-DZ8, and H-71.

51. The method of any one of claims 1-49, wherein the agent is administered by intravitreal or retro-orbital injection.

52. The method of claim 49, wherein the administration induces cellular repair of the RPE layer.

53. The method of any one of claims 1-49, wherein the method further comprises administering a vector encoding a therapeutic polypeptide.

54. The method of any one of claims 1-49, wherein the method further comprises administering a substantially purified stem cell to the subject.

55. The method of any one of claim 1-49, wherein the stem cell is administered locally by intravitreal or retro-orbital infection.

56. A pharmaceutical composition for stem cell recruitment, the composition comprising an effective amount of a small compound selected from the group consisting of geldanamycin, celastrol, 17-allylamino-17-demethoxygeldanamycin, EC102, radicicol, geranylgeranylacetone, paeoniflorin, PU-DZ8, and H-71 in a pharmaceutically acceptable excipient, the composition formulated for ocular delivery.

57. A pharmaceutical composition for stem cell recruitment in an ocular tissue, the composition comprising an expression vector comprising a polynucleotide encoding a heat shock polypeptide in a pharmaceutically acceptable excipient, the composition formulated for ocular delivery.

58. The pharmaceutical composition of claim 54, wherein heat shock polypeptide is selected from the group consisting of Hsp100, Hsp90, Hsp70, Hsp60, and Hsp40.

59. A pharmaceutical composition for stem cell recruitment in an ocular tissue, the composition comprising a polypeptide selected from the group consisting of Hsp100, Hsp90, Hsp70, Hsp60, and Hsp40 in a pharmaceutically acceptable excipient.

60. A kit comprising an effective amount of an agent that induces a heat shock response in an ocular tissue, and instructions for using the kit to increase stem cell recruitment.

61. The kit of claim 60, wherein the agent is a polypeptide, a polynucleotide, or a small compound.

62. The kit of claim 60, wherein the polypeptide is Hsp100, Hsp90, Hsp70, Hsp60, and Hsp40.

63. The kit of claim 60, wherein the polynucleotide encodes Hsp100, Hsp90, Hsp70, Hsp60, and Hsp40.

64. The kit of claim 60, wherein the small compound is selected from the group consisting of geldanamycin, celastrol, 17-allylamino-17-demethoxygeidanamycin, EC102, radicicol, geranylgeranylacetone, paeoniflorin, PU-DZ8, and H-71.

65. A method of identifying an agent that increases stem cell recruitment in an ocular tissue, the method comprising:
(a) contacting an ocular cell with a test compound;
(b) identifying an increase in the expression or activity of a heat shock polypeptide relative to an untreated ocular cell, thereby identifying a compound that increases stem cell recruitment.

66. A method of identifying an agent that increases stem cell recruitment in an ocular tissue, the method comprising:
(a) contacting an ocular cell with a test compound;
(b) identifying an increase in the number of stem cells in the tissue.
